(19) United States
(12) Patent Application Publication
(10) Pub. No.: US 2012/0276635 A1
(43) Pub. Date: Nov. 1, 2012

(54) FLUIDIC DEVICE

(75) Inventors: Chang Lu, Blacksburg, VA (US); Hsiang-Yu Wang, Hsinchu City (TW); Jun Wang, Pasadena, CA (US)

(73) Assignee: Purdue Research Foundation, West Lafayette, IN (US)

(21) Appl. No.: 13/537,182

(22) Filed: Jun. 29, 2012

Related U.S. Application Data
(63) Continuation of application No. 11/583,535, filed on Oct. 19, 2006.
(60) Provisional application No. 60/728,260, filed on Oct. 19, 2005.

(51) Int. Cl. C12N 13/00; C12N 15/02

(52) U.S. Cl. 435/450; 435/173.6

(57) ABSTRACT

A fluidic device for cell electroporation, cell lysis, and cell electrofusion based on constant DC voltage and geometric variation is provided. The fluidic device can be used with prokaryotic or eukaryotic cells. In addition, the device can be used for electroporative delivery of compounds, drugs, and genes into prokaryotic and eukaryotic cells on a microfluidic platform.

### CROSS-REFERENCE TO RELATED APPLICATIONS

This application is a Continuation application of U.S. patent application Ser. No. 11/583,535 filed Oct. 19, 2006, the entire contents of which are hereby incorporated by reference. U.S. patent application Ser. No. 11/583,535 claims the benefit of U.S. Provisional Patent Application No. 60/728,260, filed Oct. 19, 2005, the entire contents of which are hereby incorporated herein by reference.

### TECHNICAL FIELD

This invention relates to the field of fluidic devices. Specifically, the invention is directed toward devices and methods for electrical lysis, electropermeabilization and electrofusion of cells on a fluidic platform, using constant direct current (DC) voltage and geometric variation in a fluidic channel.

### BACKGROUND

Electroporation is a significant increase in the electrical conductivity and permeability of the cell plasma membrane caused by an externally applied electric field. It is usually used in molecular biology as a way of introducing some substance into a cell, such as loading it with a molecular probe, a drug that can change the cell's function, or a piece of coding DNA, to increase gene expression (Neumann et al. 1982, EMBO J. 1: 841-845). Typically, electrical pulses with defined voltages and widths are applied to cause the formation of small pores in the cell membrane. If the electrical pulses are moderate in strength and short in duration, the membrane can become transiently permeable and then reseal itself upon removal of the electric field. Increasing the strength and the duration of the electric field can lead to cell lysis and release of intracellular materials.

Cell lysis is a critical step in the analysis of intracellular contents. Biochemical analysis of cellular contents such as nucleic acids and proteins is of significant interest to the biological, medical, and pharmaceutical communities. Detection of abnormal genes and proteins in the intracellular materials provides important clues for early diagnosis of diseases.

Recently, there have been efforts to develop and manufacture microfluidic systems to perform various chemical and biochemical analyses and syntheses, both for preparative and high throughput analytical applications (Andersson and van den Berg, 2003, Sensors and Actuators B—Chemical 92: 315-325). The methods of microfluidic cell lysis can be roughly divided into four categories: chemical lysis, thermal lysis, mechanical lysis, and electrical lysis. Chemical lysis disrupts the cell membrane by mixing the cells with lytic agents such as sodium dodecyl sulfate or hydroxide. However, chemical lysis introduces lytic agents which may denature proteins and interfere with subsequent biological assays. Thermal lysis can lyse cells at high temperature (˜94° C.) prior to their DNA analysis. However, thermal lysis is not practical for protein-based assays, due to protein denaturation that occurs during thermal lysis. Mechanical forces such as microscale sonication and nanobarb filtration have been used in microfluidic devices for the purposes of cell lysis; these require the use of special devices and methods.

Electrical cell lysis has gained substantial popularity in the microfluidics community due to its application in rapid recovering of intracellular contents without introducing lytic agents (Cheng et al., 1998, Nature Biotech. 16: 541-546; McClain et al., 2003, Anal. Chem. 75: 5646-5655). Electrical cell lysis is based on electroporation, typically involving the use of pulsed electric fields. Exponentially decaying pulses or square wave pulses have been typically applied to transiently permeabilize the cell membrane. Most existent microfluidic electrical lysis devices apply alternating current or pulsed direct current electric fields. To use these methods, high density microscale electrodes or structures with subcellular dimensions need to be fabricated.

Cell fusion is a powerful tool for analysis of gene expression, chromosomal mapping, antibody production, cloning mammals, and cancer immunotherapy. Current chemical and virus-mediated cell fusion methods suffer from limitations such as toxicity to cells, batch-to-batch variability, and low efficiency. In comparison, electrofusion, which has been based on the application of electric pulses, can be applied to a wide range of cell types with high efficiency and high post-fusion viability. Electrofusion typically requires specialized equipment which generates both low-voltage AC for cell alignment/contact and high-voltage DC pulses for cell fusion (White, 1995, Electrofusion of mammalian cells, in Methods in Molecular Biology, ed. Nickoloff, J. A., Humana Press Inc., Totowa, N.J., Vol. 48, pp 283-294). Due to the complexity and cost associated with the instrumentation, few studies have explored realizing this procedure on a microfluidic platform.

Cell electropermeabilization, lysis, and electrofusion are important tools in delivery of drugs and genes which are impermeable to the cell membrane, rapid analysis of intracellular contents, bacteria sterilization, and antibody production. Fluidic techniques, and in particular microfluidics, through high throughput and parallel operations, low sample consumption, and high level of automation and integration, offer an improved platform for these applications. The invention described here addresses these and related needs.

### SUMMARY OF THE INVENTION

This invention provides a fluidic device having a flow channel defining a fluid flow path having at least two sections. The device may be a microfluidic device. The fluidic device may be used for cell permeabilization, for delivery of a molecule which is impermeant to the plasma membrane into the cell, or for gene delivery into the cell. The fluidic device also may be used for cell lysis.

In particular, this invention provides a fluidic device having a flow channel in which the flow channel comprises alternating sections of different cross-sectional area. The sections may be arranged successively, with successive sections each located downstream of preceding sections. Where the flow channel includes two sections, the cross-sectional area of the flow channel in the direction of fluid flow decreases from one section to another section, such that upon application of a constant direct current voltage across the flow channel, the electric field intensity in downstream section is greater than the electric field intensity in the upstream section.

The flow channel may include further sections of varying cross-sectional area. For example, the flow channel may include three sections or area. In this example, the first or upstream area or section has a cross-sectional area, the second or middle area, which is downstream of the first section, has cross-sectional area that is smaller than the area of the first area or section, and the third section or area, which is downstream of the middle section or area, has a cross-sectional area that is larger than the second or middle section. In this example, the middle section or area may be narrower than both the first and second sections or areas.

Additional sections of alternating cross-sectional area also may be provided, where each section has a greater or lesser cross-sectional area than that of the preceding section. In one example, the sections may be stepped down, or up as the case may be. In another example, the fluid flow channel may be tapered from one section to another where the cross-sectional area of the channel narrows from an upstream part to a downstream part. Successive parts may be provided where the channel widens and then again tapers.

The fluidic device may be used for cell electroporation. Thus, a method of cell electroporation also is provided, where at least one cell is subjected to a constant electric field. Where the device is used for cell electroporation, the electric field intensity in one of the sections of the flow channel having a smaller cross-sectional area than a preceding section of the channel is greater than the electric field intensity threshold for cell electroporation. The method of cell electroporation may be used for cell permeabilization, delivery of a molecule which is impermeant to the plasma membrane into the cell, or for gene delivery into the cell. Alternatively, the method of electroporation may be used for cell lysis.

The fluidic device also may be used for electrofusion of at least two cells, where the at least two cells are subjected to a constant direct current voltage field. Where the device is used for electrofusion, the electric field intensity in one of the sections of the flow channel having a smaller cross-sectional area than a preceding section of the channel is greater than the electric field intensity threshold for electrofusion of the at least two cells.

### BRIEF DESCRIPTION OF THE DRAWINGS

FIG. 1 is a perspective view of a fluidic device.

FIG. 2 illustrates a partial schematic view of a flow channel of an exemplary fluidic device, in which the cross-sectional area of the channel decreases from a first section to a second section.

FIG. 3 illustrates another partial schematic view of a flow channel of an exemplary fluidic device, in which the cross-sectional area of the channel decreases from a first section to a second section and then increases.

FIG. 4 illustrates another partial schematic view of a flow channel of an exemplary fluidic device, where the fluid flow channel has multiple sections with varying cross-sections.

FIG. 5 illustrates another partial schematic view of a fluidic device. FIG. 5(a) shows a fluidic device with receiving and sample reservoirs attached. FIG. 5(b) is a microscopic image of a part of the device showing the reduction in width of the flow channel.

FIG. 6 illustrates another partial schematic view of a flow channel of an exemplary fluidic device, where the fluid flow channel tapers.

FIG. 7 illustrates another partial view of the flow channel of an exemplary fluidic device, where the fluid flow channel tapers and then widens.

FIG. 8 depicts graphs showing the relationship between the applied voltage and the number of viable cells in the receiving reservoir for devices with three different configurations.

FIG. 9 depicts graphs showing the velocity and the duration of exposure to the electric field for cells in different sections of fluidic devices with different configurations.

FIG. 10 is a graph depicting the percentage of lysed CHO-K1 cells as a function of the electric field strength in a narrower section of the flow channel.

FIG. 11 depicts graphs showing the effects of electric field strength on CHO-K1 cell permeability and viability, as established via delivery of SYTOX Green into cells.

FIG. 12 depicts graphs showing the effects of configurations, strength, and duration of electric field on transfection of CHO-K1 cells.

FIG. 13 shows images of cells processed in a fluidic device: (a) phase contrast image of a group of electrofused cells; (b) fluorescence micrograph of the same group of cells stained with Hoechst 33342.

FIG. 14 shows graphs depicting the fusion index (a) and the relative number of viable cells (b) as a function of the electric field strength.

### DETAILED DESCRIPTION OF THE PREFERRED EMBODIMENTS

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed., 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale and Marham, THE HARPER COLLINS DICTIONARY OF BIOLOGY (1991). As used herein, the following terms have the meanings ascribed to them unless specified otherwise.

A “flow channel” refers generally to a flow path through which a solution can flow.

The term “constant direct current voltage” refers to the voltage of constant magnitude over time, which is typically generated by a direct current power supply.

“Electroporation” or “electropermeabilization” refers to a significant increase in the electrical conductivity and permeability of the cell plasma membrane caused by an externally applied electric field.

The phrase “electric field intensity threshold for electroporation” refers to the strength of an electric field that will cause pores to form in the plasma membrane. Typically this occurs when the voltage across a plasma membrane exceeds its dielectric strength. If the strength of the applied electric field and/or duration of exposure to it are properly chosen, the pores formed by the electrical pulse reseal after a short period of time, during which extracellular compounds have a chance to enter into the cell. However, excessive exposure of live cells to electric fields can cause apoptosis and/or necrosis—the processes that result in cell death. Electroporation is usually used in molecular biology as a way of introducing some substance into a cell, such as loading it with a molecular probe, a drug that can change the cell's function, or a piece of coding DNA. Electroporation with increased strength and/or duration of the electric field can lead to cell lysis and release of cellular materials.

“Permeability” is a measure of the ability of a membrane to transmit fluids. As used herein, increasing “cell permeabilization” refers to increasing the transmission of fluids and various molecules through the cell membrane (plasma membrane).

“Cell fusion” refers to the melding of two or more cells into one cell. “Electrofusion” as used herein refers to cell fusion under the influence of an electric field.

The phrase “electric field intensity threshold for cell fusion” refers to the strength of an electric field that will cause fusion of at least two cells. A number of different fluidic devices having unique flow channel architectures are provided here, as well as methods for using the devices to conduct a variety of high throughput assays and analyses. The fluidic device may be used for cell permeabilization, for delivery of a molecule which is impermeant to the plasma membrane into the cell, or for gene delivery into the cell. The fluidic device also may be used for cell lysis.

In one example, the fluidic device is used for flow-through electroporation of cells based on applied constant direct current (DC) voltage. The fluidic device uses constant direct current electric field to provide for high throughput cell electropermeabilization, cell lysis, or cell electrofusion. The cells may be either prokaryotic or eukaryotic. When the volumes of fluids used are small, in the microliter and/or nanoliter range, the fluidic device may be a microfluidic device.

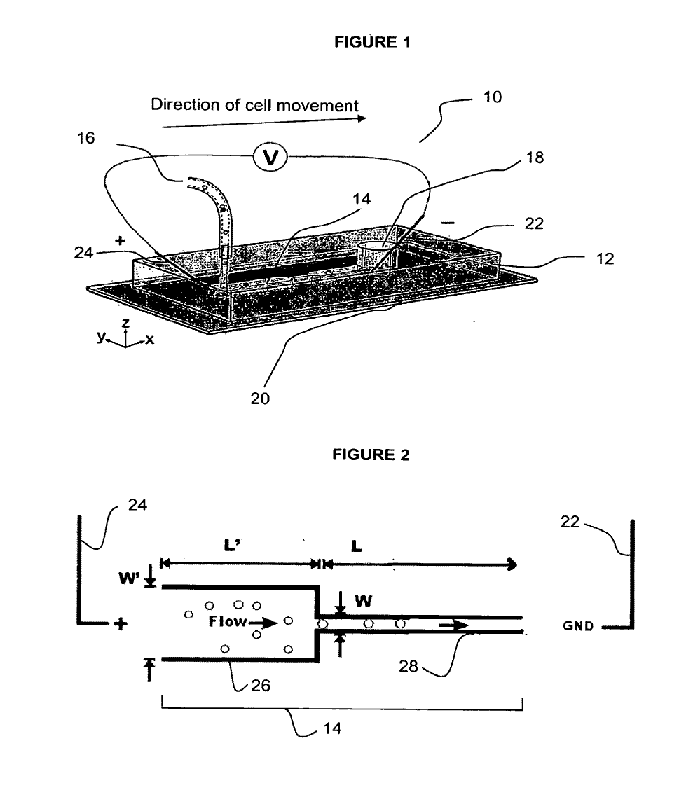

FIG. 1 shows a perspective view of a fluidic device 10. The device 10 may include a substrate 12. A flow channel 14 may be formed in the substrate. The device 10 may further include an input port 16 or reservoir for introducing cells into the flow channel 14. The device may optionally include a receiving reservoir 18 for collecting cells that have passed through the flow channel 14, which may be located in fluid communication with the flow channel 14, as shown in FIG. 1. After the cells have passed through the flow channel 14, they may be collected in the receiving reservoir 18.

The fluidic device optionally may include a support 20. The fluidic device 10 may be hermetically sealed to the support 20. The support 20 may be manufactured of essentially any material, although the surface should be flat to ensure a good seal, as the seal formed is primarily due to adhesive forces. Examples of suitable supports include glass, plastics and the like. For example, the support 20 may be a glass slide, as shown in FIG. 1.

A negative (−) ground) electrode 22 and a positive (+) electrode 24 may be used for application of an electric field across the flow channel 14. Various types of electrodes may be used as are known. For example, Pt/Au wires or deposited metal layers on the substrate may be used as electrodes. Cells may be loaded into a sample input port 16 and transported through the flow channel 14 to a receiving reservoir. Optionally, cell may first be loaded into a sample reservoir that is in fluid communication with the flow channel 14. As shown in FIG. 1, the positive electrode 24 may be in the vicinity of the receiving reservoir 18 and the negative electrode 22 may be in the vicinity of the input port 16 or a sample reservoir. Alternatively, the positive electrode 24 may be in the vicinity of the input port 16 and the negative electrode 22 or ground may be in the vicinity of the receiving reservoir 18. One skilled in the art will know that various types of power supplies or batteries can be used to generate constant DC voltage.

The flow channel 14 may define a fluid flow path having at least two sections, where the sections have different cross-sectional areas. As shown in FIG. 2, the fluid flow channel 14 may have a first section 26 having a larger cross-sectional area than the cross-sectional area of a second section 28 downstream of the first section 26. The first section 26 may be described as the wide section or wider section and the second section 28 may be described as the narrow or narrower section.

The sections may be arranged successively, with successive sections each located downstream of preceding sections. Where the flow channel includes two sections, the cross-sectional area of the flow channel in the direction of fluid flow decreases from one section to another section, such that upon application of a constant direct current voltage across the flow channel, the electric field intensity in downstream section is greater than the electric field intensity in the upstream section.

The flow channel may include further sections of varying cross-sectional area. For example, the flow channel may include three sections or area. In this example, the first or upstream area or section has a cross-sectional area, the second or middle area, which is downstream of the first section, has cross-sectional area that is smaller than the area of the first area or section, and the third section or area, which is downstream of the middle section or area, has a cross-sectional area that is larger than the second or middle section. In this example, the middle section may be narrower than both the first and section sections.

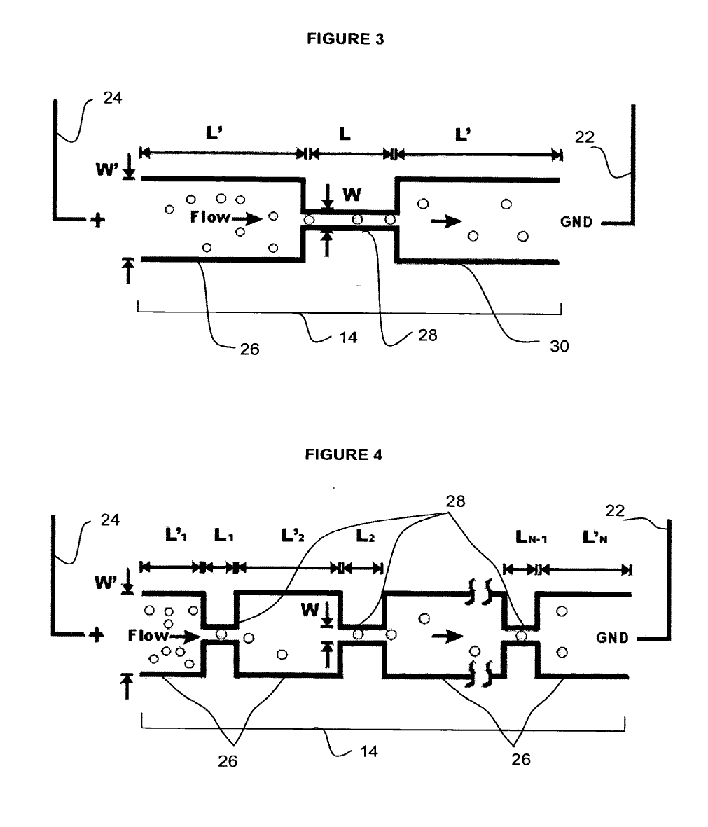

Additional sections of alternating cross-sectional area also may be provided, where each section has a greater or lesser cross-sectional area than that of the preceding section. For example, as shown in FIG. 3, the channel 14 may include three sections 26, 28, 30, where a third section 30 is downstream of the second section 28. As shown, the third section 30 may be wider and, thus, have a greater cross-sectional area than the second section 28. As shown in FIG. 4, the channel 14 may be configured to include multiple wide 26 and multiple narrow 28 sections, arranged successively, where the wide sections 26 and narrow sections 28 alternate. As shown in FIGS. 3 and 4, cells flow successively from the first wide section through the successive narrow and wide sections.

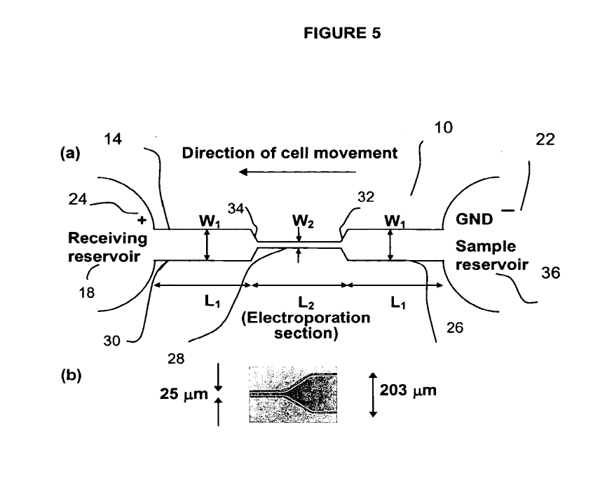

FIG. 5(a) is a schematic illustrate of a fluidic device 10 having two sections of larger cross-sectional area and a middle section having a smaller cross-sectional area. In this example, cells are introduced from a sample reservoir 36 and move successively through the sections 26, 28, 30, to the receiving reservoir 18. Thus, in the configurations shown in FIG. 5, the cross-sectional area of the flow channel 14 first decreases and then increases.

FIG. 5(b) depicts a microscopic image of a part of the device showing the reduction in width of the flow channel. In this example, the reduction in width is from 203 μm in the wide section of the flow channel to 25 μm in the narrow section of the flow channel.

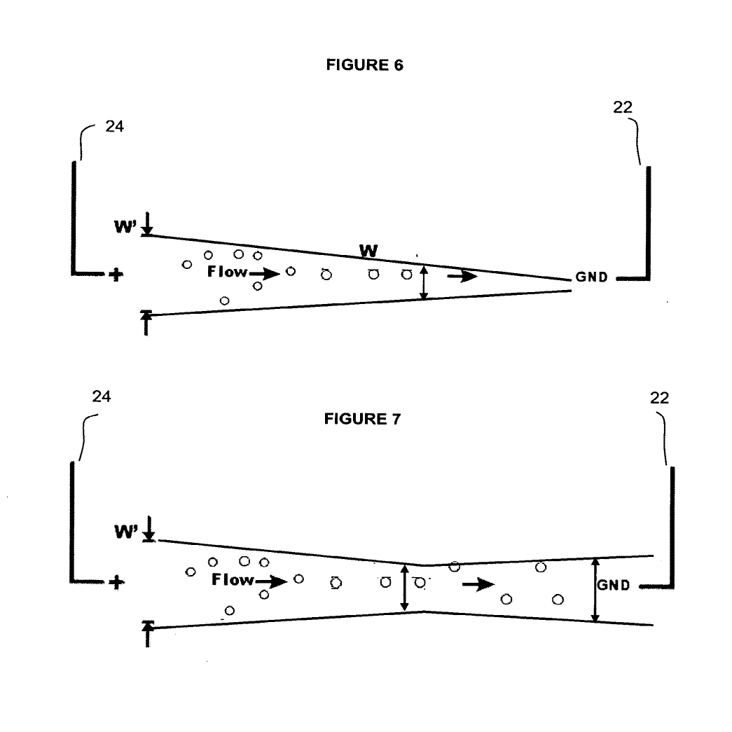

As shown in FIGS. 3 and 4, the change in cross-sectional area may be abrupt or, as shown in FIG. 5, the first wide section may have a transition zone 32 that more gradually narrows to the second section. Similarly, as shown, the second narrow section 28 may have a transition zone 34 that may more gradually widen from the narrow section. In another example, shown in FIGS. 6 and 7, the fluid flow channel may be tapered from one section to another where the cross-sectional area of the channel narrows from an upstream part to a downstream part. As shown in FIG. 7, successive sections may be provided where the channel tapers and then again widens.

As shown in the FIGS. 1-4 and 6-7, under the influence of the electric field generated, for example, by a DC power supply the cells flow through the channel going in the direction from the positive electrode (+) 24 toward the negative electrode (−) 22. As shown in FIG. 5, under the influence of the electric field generated, for example, by a DC power supply, the cells flow through the channel going in the direction from the negative (ground) electrode 22 (−) toward the positive electrode (+) 24. Alternatively, the flow of cells through the channel in either direction can be controlled using pressure, for example generated by a syringe pump.

The fluidic device 10 may be fabricated using various materials, e.g. polydimethylsiloxane (PDMS), using methods known in the art (Duffy et al., 1998, Anal. Chem. 70: 4974-4984). Examples of suitable substrate materials in which the channel and other parts can be formed include polymers, copolymers, elastomer, ceramic, quartz, silicon, silicon dioxide, silica, glass, or mixtures thereof.

The fluidic device 10 may be constructed at least in part from elastomeric materials and constructed by single and multilayer soft lithography (MSL) techniques and/or sacrificial-layer encapsulation methods. The basic MSL approach involves casting a series of elastomeric layers on a micro-machined mold, removing the layers from the mold and then fusing the layers together. In the sacrificial-layer encapsulation approach, patterns of photoresist are deposited wherever a channel is desired. These techniques and their use in producing microfluidic devices are discussed in detail, for example, by Unger et al., 2000, Science 288:113-116; U.S. Pat. No. 7,118,910; and PCT Publication WO 01/01025), each of which is incorporated by reference in their entireties here. The material used does not alter the principles under which the fluidic device operates.

In one example, a fluidic device may be fabricated using PDMS as a substrate, and using standard soft lithography method. The microscale patterns can be created using computer-aided design software, e.g. FreeHand M X, Macromedia, San Francisco, Calif., and then printed on high-resolution (5080 dpi) transparencies. Transparencies can be used as photomasks in photolithography on a negative photoresist (SU-8 2025, MicroChem. Corp., Newton, Mass.). The thickness of the photoresist and hence the depth of the flow channels can be varied according to the desired application. In one example, the flow channel depth is in the micrometer range, i.e. 1-1,000 μm.

The channel depth can be measured, e.g., using a Sloan Dektak3 ST profilometer. The pattern of channels in the photomask is then replicated in SU-8 after exposure and development. The fluidic channel and the desired sections can be molded by casting a layer (˜5 mm) of PDMS prepolymer mixture (General Electric Silicones RTV 615, MG chemicals, Toronto, Ontario, Canada) with a mass ratio of A:B=10:1 on the SU-8/silicon wafer master treated with tridecafluoro-1,1,2,2-tetrahydrooctyl-ltrichlorosilane (United Chemical Technologies, Bristol, Pa.). The prepolymer mixture is then cured at 85° C. for 2 hours in an oven and then peeled off from the master. Glass slides 20 are cleaned in a basic solution (H₂O: NH₄OH (27%):H₂O₂ (30%)=5:1:1 volumetric ratio) at 75° C. for an hour and then rinsed with DI water and blown dry. The PDMS chip and the pre-cleaned glass slide are oxidized using a Tesla coil (Kimble/Kontes, Vineland, N.J.) in atmosphere. The PDMS chip is immediately brought into contact against the slide after oxidation to form closed channels.

The devices formed according to the foregoing method result in a type of substrate (e.g., glass slide) forming one wall of the flow channel. Alternatively, the device once removed from the mother mold may be sealed to a thin membrane (e.g. elastomeric material) such that the flow channel is totally enclosed in the material. The resulting device may then optionally be joined to a substrate support, as previously discussed.

The geometric configuration of the fluidic device and, in particular the configuration of the flow channel is used to locally amplify the electric field in a predetermined section of the flow channel, so that the electric field intensity is above the threshold for electropermeabilization, lysis, or electrofusion. In the rest of the channel, the electric field remains well below the threshold field intensity for electropermeabilization, lysis, or electrofusion, so that the cells are only transported.

Since long exposure to strong electric field can lead to cell death, geometrical modifications can be used to localize the electric field in defined sections in a fluidic channel, thereby minimizing the cell exposure to the electric field. Based on Ohm's law, when a DC voltage is applied at a conductor (e.g., a buffer-filled channel) the potential drop at individual sections of the conductor is proportional to its resistance within the section. When the depth is uniform in a fluidic channel, the local field strength E is inversely proportional to the width of the channel within the section W. The overall voltage needed for operation of the device is substantially lower than that needed by a channel without the special geometry.

The cells may be electroporated while flowing through the geometrically defined narrow electroporation section. Cells may be electroporated under constant DC voltage with a high survival rate. The device is suitable for electropermeabilization of both prokaryotic and eukaryotic cells.

The fluidic device also may carry out high throughput electrical cell lysis in a constant electric field. In one example, the electric field is constant direct current field. Cell lysis is thus made possible in a DC field without introducing bubbles and electrolysis of water.

The device may be useful as a cell biology tool which can be easily incorporated with other analytical methods. For example, the integration of cell lysis and analytical tools such as electrophoresis provides for analysis of cellular contents of interest to the biological, medical, and pharmaceutical communities.

The fluidic device is suitable for high throughput electropermeabilization of prokaryotic and eukaryotic cells and it can be easily arranged in high density arrays for screening of drugs and genes. Systems utilizing the fluidic device may provide for high throughput, low sample amount, and high level of automation and integration in drug discovery, gene therapy, and functional genomics. It may further facilitate the delivery of libraries of small molecules and genes into cells, for screening of their functions on a fluidic platform. When small volumes of fluid are used (in the micro- and nano-scale range), the platform is microfluidic.

The fluidic device also may be used for cell electrofusion using a common DC power supply on a fluidic platform. In principle it is possible to control the overall voltage so that only the field in the narrow section(s) is high enough for cell fusion and the field in the rest of the channel is too weak to have adverse effects on the cell viability. When cells flow through the device, they experience field intensity variations equivalent to electrical pulse(s). The equivalent of the “pulse width” is determined by the length of the narrow section and the velocity with which the cells move through the narrow section.

The device and electrofusion method can be used for fusion of one type of cells. One skilled in the art will know that the device and method can be used to fuse two or more cells, or two or more different cell types and thus obtain hybrid cells or chimeric cells, while generating prokaryotic fusions, eukaryotic fusions, or combinations thereof.

The fluidic device can handle a number of cells with high throughput. Because the absolute values of the geometry are not critical, the channel size can be much larger than cell dimensions, e.g. in the case of prokaryotic cells, to avoid clogging and adsorption.

The design of the fluidic device is superior to using a fluidic channel with a uniform width. For example, the narrow sections can be fabricated to be very short, which enables for short exposures with cells having reasonable flow rates through the channel.

The instrumentation used is extremely simple and safe. A DC power supply is used to apply the electric field and simple fluidic channels will generate alternating high and low fields by geometric modifications. Many applications require the use of less than 100 Volts (V). This eliminates the danger and inconvenience of using a high voltage electropulsator on a fluidic platform.

### Design and Fabrication of the Fluidic Device

Electroporation experiments are typically carried out using specialized capacitor discharge equipment to generate electrical pulses with defined intensities and durations (electropulsation). In contrast, in the present design, constant DC voltage is applied to generate alternating high and low fields inside a fluidic channel with geometric variations. The geometric variations refer to different cross-sectional areas in different (wide and narrow) sections of the channel. The cells are passed through the device so that, as they pass through the wide and narrow sections, they experience electric field variation similar to that of electrical pulses. The field strengths in the wide sections (E′) and the narrow sections (E) will roughly have the following relationship with the channel widths in the wide section (W′) and in the narrow section (W): E′/E=W/W′. The accurate field intensity distribution in the device can be computed using software.

The electric field variation effect does not depend on the absolute dimensions of the channel but instead is related to the relative sizes of the different channel sections, narrow section(s) and wide section(s). This geometric variation approach is demonstrated in the examples section below based on microfluidic channels, due to their ease of fabrication; however, the same principle also applies to systems with larger dimensions when the ratio in the cross-section of the wide sections to narrow sections is kept.

General information regarding the design and fabrication of the device can be found in Wang and Lu, 2006, Anal. Chem. 78: 5158-5164; Wang and Lu, 2006, Biotechnology and Bioengineering, DOI:10.1002/bit.21066, in press; Wang et al., 2006, Biosensors and Bioelectronics, DOI:10.1016/j.bios.2006.01.032, in press), incorporated by reference herein.

As discussed above, FIG. 3 has a flow channel 14 with one narrow section (middle) 28 alternated with two wide sections 26 and 30. FIG. 4 shows a flow channel 14 that has (N−1) narrow sections alternated with N wide sections, where N is an integer larger than 2. The direction of the cell flow is from left to right, i.e. from a +labeled reservoir (with the positive electrode 24 in) toward the ground (GND) labeled reservoir (with the ground electrode 22 in). The device of FIG. 3 provides cells with a single exposure to the high electric field in the narrow section. The field in the narrow section is designed to be higher than the threshold for the desired application, e.g. electroporation or cell lysis or electrofusion. The device of FIG. 4 provides multiple exposures to the high electric field, each exposure in one of the N−1 narrow sections of the device. The two configurations are analogous to having one (FIG. 3) or N−1 (FIG. 4) electrical pulses in the case of using electropulsation. As set forth above, the wide sections and the narrow sections can be delineated in a step-down fashion, as is schematically shown in FIGS. 2-5, have tapered transition zones (FIG. 7) or form a continuous taper (FIG. 6). Cells will experience low/high electric field when they flow under pressure through the channel's wide/narrow sections, respectively. A skilled artisan can select the geometry (cross-section and length) of the sections in the channel, the velocity with which cells move (flow) through the sections, and the overall DC voltage in a way that cell electroporation/lysis/electrofusion occurs only in the narrow sections of the fluidic device.

The speed for processing cells using the fluidic device depends on the cell concentration, the flow rate, and the dimensions of the device. In a channel of the microfluidic device, the speed can be up to hundreds of cells per minute. The durations for the cell to stay in the fields will vary with different applications. The length of cell stay in a field of particular strength is determined by the velocity of the cell flow and the lengths of the sections. To alleviate the effect of Joule heating, the buffer used for electroporation can contain an osmoticum (e.g. sucrose) as a gradient to maintain the osmotic pressure balance with a low ionic strength. Alternatively, the buffer can be internally or externally cooled to prevent or minimize heating.

### Electric Field Strength

Like any conductor, the resistance within a certain section of a fluidic channel is determined by the conductivity, the length, and the channel's cross-sectional area. For a channel with uniform depth and a varying width as shown in FIGS. 1-7, the field strength (E) is different in different sections. According to Ohm's law, the electric field strength (E₁) in the wide section (W₁) and the electric field strength (E₂) in the narrow section (W₂) can be closely approximated using the below equations, when the lengths (L) of the wide and the narrow sections are the same.

$$\begin{matrix}{E}_{1} = \frac{V}{L ( 2 + \frac{{W}_{1}}{{W}_{2}} )} & ( 1 ) \\ {E}_{2} = \frac{V}{L ( 2 + \frac{2 {W}_{2}}{{W}_{1}} + 1 )} & ( 2 ) \\ {E}_{2} / {E}_{1} = {W}_{1} / {W}_{2} & ( 3 )\end{matrix}$$

The fluidic device may be designed with the width of the narrow section W₂ being much smaller than width of the wide section W₁. This design results in much higher field strength in the narrow section(s) compared to that of the wider section(s) when a DC field is applied across the whole length of the device. Similar geometric modifications have been shown to create local electric field as high as 10⁵V/cm without causing water electrolysis and boiling (See for example, Jacobson et al., 1998, Anal. Chem. 70: 3476-3480, Plenert and Shear, 2003, Proc. Natl. Acad. Sci. USA 100: 3853-3857, incorporated by reference here).

Modeling of the electric field in the device may be done in a variety of ways. For example, one skilled in the art can apply the Conductive Media DC model from Cornsol 3.2 (COMSOL, Inc., Burlington, Mass.) to model the electric field distribution. Assuming there is no ion concentration gradient in the flowing fluid carrying the current, Ohm's law can be used for current density calibration.

The constant electric field can be generated in a variety of ways. A direct current power supply can generate constant direct current field by supplying a voltage with a constant value over time.

The W₁/W₂ ratio may be increased to adapt the device for electroporation of different types of cells. In the experiments conducted and described below, the choice of W₁ was limited by the maximum feature size that did not cause the channel to collapse. W₁ can be increased, for example, by having supporting structure in the wide sections or by simply increasing the depth of the channel. The smallest W₂ was determined by the resolution allowed by soft lithography. A smaller W₂ can be achieved by using more advanced lithography techniques.

Using the geometry chosen for the fluidic device of this invention, when the electric field intensity in the narrow section E₂ reaches the threshold for electroporation, the electric field in the wide section E₁ is well under the threshold for electroporation.

In another example, using the geometry chosen for the fluidic device of this invention, when the electric field intensity in the narrow section E₂ reaches the threshold for cell lysis, the electric field in the wide section E₁ is well under the threshold for cell lysis.

In yet another aspect, using the geometry chosen for the fluidic device of this invention, when the electric field intensity in the narrow section E₂ reaches the threshold for cell electrofusion, the electric field in the wide section E₁ is well under the threshold for cell electrofusion.

### EXAMPLES

The invention will be further described by reference to the following detailed examples. These examples are provided for purposes of illustration only, and are not intended to limit the claimed invention.

### Fluidic Device Fabrication

Fluidic devices (microchips) were fabricated based on PDMS using standard soft lithography method (Duffy et al., 1998). The microscale patterns were first created using computer-aided design software (FreeHand MX, Macromedia, San Francisco, Calif.) and then printed out on high-resolution (5080 dpi) transparencies. The transparencies were used as photomasks in photolithography on a negative photoresist (SU-8 2025, MicroChem. Corp., Newton, Mass.). There could be up to 5% error introduced to the width of the channel due to the quality of the photomask. The thickness of the photoresist and hence the depth of the channels was around 33 μm (measured by a Sloan Dektak3 ST profilometer). The pattern of channels in the photomask was replicated in SU-8 after exposure and development.

The microfluidic channels were molded by casting a layer (˜5 mm) of PDMS prepolymer mixture (General Electric Silicones RTV 615, MG chemicals, Toronto, Ontario, Canada) with a mass ratio of A:B=10:1 on the SU-8/silicon wafer master treated with tridecafluoro-1,1,2,2-tetrahydrooctyl-ltrichlorosilane (United Chemical Technologies, Bristol, Pa.). The prepolymer mixture was cured at 85° C. for 2 hours in an oven and then peeled off from the master. Glass slides were cleaned in a basic solution (H₂O: NH₄OH (27%):H₂O₂ (30%)=5:1:1 volumetric ratio) at 75° C. for an hour and then rinsed with DI water and blown dry. The PDMS chip and the pre-cleaned glass slide were oxidized using a Tesla coil (Kimble/Kontes, Vineland, N.J.) in atmosphere. The PDMS chip was immediately brought into contact against the slide after oxidation to form closed channels.

### Electroporation of Escherichia Coli Cells

Green Fluorescent Protein (GFP)-expressing Escherichia coli transformed by pQBI T7-GFP plasmid (Qbiogene, Irvine, Calif.) were used in the cell lysis experiments. These cells were cultured in Luria-Bertani (LB) broth (BIO 101 Systems, Irvine, Calif.) with 50 μg/ml of ampicillin at 37° C. for 16 hours.

Approximately 1 ml of the culture was centrifuged and the LB broth was removed. The cells were resuspended in 1 ml phosphate buffer (1.35 mM KH₂PO₄, 2 mM Na₂HPO₄, 0.05% Tween 20, pH 7.0). The cell density after the resuspension was around 10⁸-10⁹ cells/ml. The resulting suspension was diluted with phosphate buffer to about 10⁶ cells/ml and then loaded into the chip. Tween 20 was added to decrease the adsorption of cells and the intracellular contents to the channel walls.

The design and some different configurations of the fluidic device of this invention, used for cell electroporation, lysis, and cell electrofusion are described in FIGS. 1-7 and in Table 1. The cell suspension, containing a concentration of about 10⁶ cells/ml phosphate buffer, was loaded into the sample reservoir. The channel and the receiving reservoir were filled with the phosphate buffer described above. Both reservoirs contained about 30 μl liquid at the beginning of each experiment.

Microfluidic devices with three different configurations (A, B, and C; Table 1) were tested. Configurations A, B, and C had different W₁/W₂ ratios (8.1, 6.4, and 1.9, respectively,) and the single section length L was 5.0 mm for configuration A and 2.5 mm for configurations B and C.

TABLE 1
Different configurations of microfluidic devices

| Configuration | W₁ (μm) | W₂ (μm) | L (mm) |
|---|---|---|---|
| A | 203 | 25 | 5.0 |
| B | 212 | 33 | 2.5 |
| C | 219 | 115 | 2.5 |

The design of the device was able to considerably lower the total voltage needed to generate a high field intensity compared to a microfluidic channel without geometric modification. For example, for a device with B configuration, the total voltage V needs to be about 500 V to generate 1500 V/cm field intensity in the narrow section, i.e. 1 V generates about 3 V/cm in E₂. In absence of geometric modification, the total voltage would have to be 1125 V to generate the same field strength.

An electric field was then established along the length of the device by inserting two platinum wires into the reservoirs with the ground end in the cell sample reservoir and the positive end in the receiving reservoir. The voltage was provided by a high voltage power supply (PS350, Stanford Research Systems, Sunnyvale, Calif.). The bacterial cells were flowing through the device under the influence of the electric field once the voltage was on.

The duration of each test was 20 minutes. After switching off the voltage, solutions in both reservoirs were recovered for plate count to record the numbers of viable cells. Cells were collected using a pipette, streaked onto LB agar plates and incubated overnight at 37° C. for colony counting. To facilitate the comparison of plate count results, the amount of viable cells in the receiving reservoir was indicated as relative numbers by designating the number of viable cells at the lowest voltage (285V for configuration A and 185V for configurations B and C) to be 1.

When the experiment was started, the sample reservoir typically contained about 10⁴ cells. Depending on the voltage and the device configuration, 300-5,000 cells passed from the sample reservoir to the receiving reservoir during the course of the experiment (20 minutes). Devices of different configurations were tested under varying voltage.

In principle, the amplification effect can be further enhanced by, for example, either decreasing only the length of the narrow section or increasing W₁/W₂. For example, when the length of the narrow section is decreased to 1 mm in configuration B, 1 V in the overall voltage is able to contribute about 5.6 V/cm to E₂.

The relationship between the overall voltage applied between the two reservoirs and the viability of cells after flowing through the devices of different configurations was determined in various experiments. The irreversible disruption of cell membrane by electroporation was the main reason for the loss of cell viability (or the ability to form a colony post-electroporation treatment).

The behavior of GFP-expressing E. coli in the channel was observed using a fluorescence microscope. The fluidic device was mounted on inverted fluorescence microscope (1X-71, Olympus, Melville, N.Y.) with a 20× dry objective (NA=0.40). Epifluorescence excitation was provided by a mercury lamp, together with bright field illumination. The excitation and emission were filtered by a fluorescence filter cube (Exciter HQ480/40, emitter HW535/50, and beam splitter Q5051p, Chroma technology, Rockingham, Vt.). Images were taken with a CCD camera (ORCA-285, Hamamatsu, Bridgewater, N.J.) at a frame rate of 10 Hz.

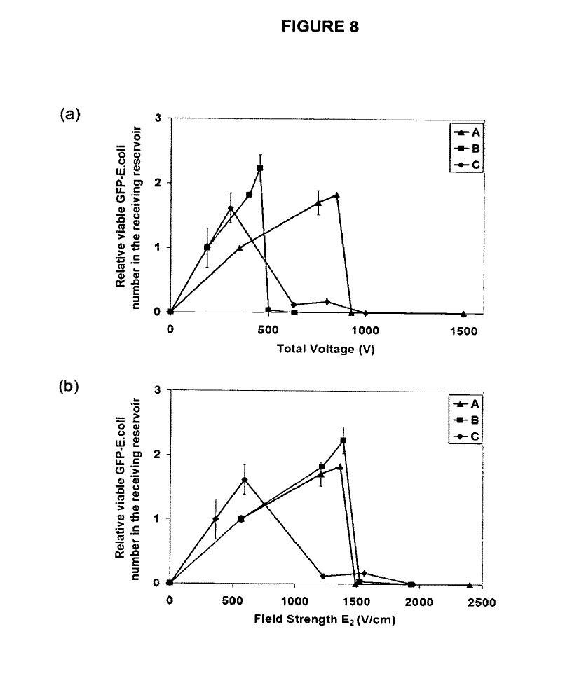

The viable cells in the sample and receiving reservoirs after the lysis experiment were counted using plate count (FIG. 8). FIG. 8(a) shows the relationship between applied voltage and the number of viable cells in the receiving reservoir for devices with configurations A, B and C. FIG. 8(b) shows the relationship between the field strength in the narrow section (the electroporation/lysis section) E₂ and the number of viable cells in the receiving reservoir for devices with configurations A, B and C. Each data point was based on results from three separate tests.

FIG. 8(a) shows an initial increase in the number of viable cells when the voltage increased. Such increase in the number of viable cells was due to higher velocity of cells when the field intensity went up. After the near-linear increase in the lower voltage regime, the number of viable cells experienced a rather abrupt drop to zero (or close to zero) for all three configurations when the voltages went beyond certain values (930V for configuration A, 500V for configuration B, and 630V for configuration C). The data suggested that once the threshold field strength was met, nearly all the cells flowing through the device were lysed.

In FIG. 8(b), the number of viable cells was plotted against the calculated values of the electric field strength in the narrow section of the channels. The correlation in the threshold field strengths with different configurations was fairly good. In the devices of configurations A and B, cell lysis started when the field strength in the lysis section (narrow section) increased to 1500 V/cm. In devices with configuration C, the cells were substantially lysed (˜95%) when the field intensity was around 1000-1200 V/cm.

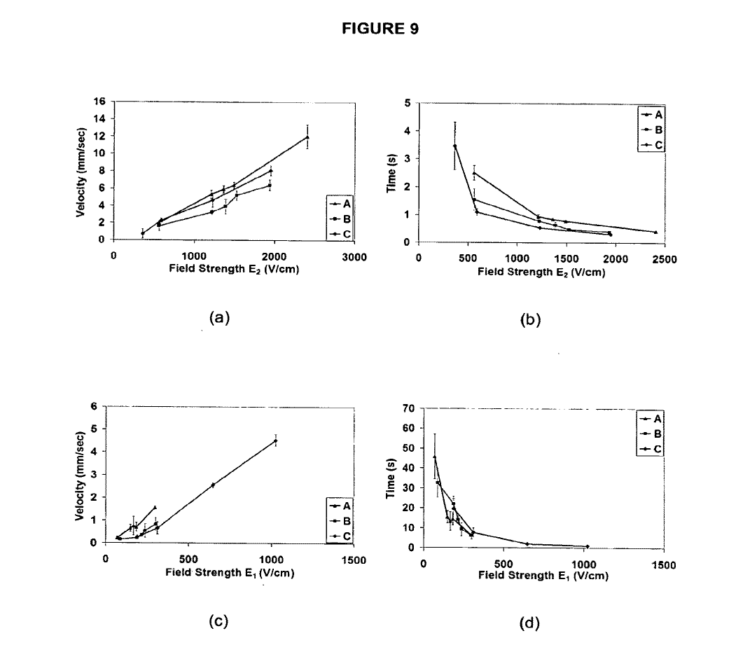

The velocity with which the cells moved through the channel was calculated based on the change in the physical location of the same cell in consecutive images and the time interval between the images. When the cell velocity was too high to observe the same cell in the next image, the length of the trail left by a cell in one image and the exposure time were used to determine the velocity. About 10-20 cells were sampled for each data point in the velocity curves shown in FIG. 9.

### Lysis of Escherichia Coli Cells

This invention provides devices and methods for single cell lysis. The fluidic device of this invention was used for lysis of green fluorescent protein (GFP)-expressing E. coli cells. Bacterial cells such as E. coli require threshold field strength for lysis significantly higher than that required by typical mammalian cells (Lee and Tai, 1999, Sens. Actuators A: Phys. 73: 74-79). Furthermore, bacterial cells are typically of much smaller sizes compared to mammalian cells. Although single cell analysis based on intracellular materials from individual mammalian cells has become standard in the literature, similar practice based on lysate from single bacterial cells has yet to be achieved (Meredith et al., 2000, Nature Biotechnol. 18: 309-312; Hu et al., 2004, Anal. Chem. 76: 4044-4049).

Different combinations of lengths and widths for different sections of the fluidic device were used. The suspension of bacterial cells with a concentration of about 10⁶ cells/int was loaded into the sample reservoir and the electric field was established for a period of time. The bacterial cells flowed through the channel to the receiving reservoir due to their own intrinsic electrophoretic mobility (electroosmotic flow was weak). The number of viable cells in the receiving reservoir after the treatment was measured using plate count. When the voltage between the two reservoirs increased, the number of viable cells in the receiving end first increased due to increased flow rate of cells and then experienced a rather abrupt drop to zero due to cell death in the strong electric field.

The onset of cell death was determined by the field strength in the narrow section. The threshold for the irreversible electroporation was around 1500 V/cm which was significantly lower than what has been reported using electropulsation (˜7000 V/cm; Lee and Tai, 1999, Sensors and Actuators A: Physical. 73: 74-79). The strength of the low field E₁ was around 190 V/cm when cell death occurred. Low field strength (<300 V/cm) of extended period (30-40 seconds) did not appear to affect the cell viability. Cells were in either E₁ or E₂ for 600-700 ms in E₂ and for several seconds in E₁.

Further details about the electrical lysis were revealed by observing GFP-expressing E. coli cells using fluorescence microscopy at the entrance and the exit of the narrow section of a device with configuration A. Images were taken with a total voltage of 1500V (2400 V/cm in the narrow section) and with a total voltage of 350V (560 V/cm in the narrow section). Higher cell traffic was observed at higher field strength. Due to the higher cell velocity, the images of E. coli cells were elongated in the direction of their movement. When E₂ was 2400 V/cm, high density of cells was observed at the entrance and no fluorescent cells were observed at the exit of the lysis (narrow) section. Upon passing through the narrow section, the cells were completely disintegrated and the intracellular contents were released into the buffer.

Cells were lysed exclusively in the narrow section. The threshold field strength for cell lysis was determined to be about 1500-2000 V/cm. Based on the analysis of cell images, it is possible—though not essential—that lysis happened by generating small but irreversible pores in the membrane instead of completely rupturing the membrane.

### Exposure of Cells to the Electric Field

The duration for cells to be exposed to the electric field is an important parameter for practicing the method of this invention. To characterize the duration of exposure to the electric field, the relationship between the velocity of cells and the field strength in different sections of the devices with configurations A, B, and C (see Table 1) was established (FIG. 9). The velocity of cells in an electric field was mainly determined by the electrophoretic mobility of cells and the eletrophoretic mobility of electroosmotic flow (EOF). Since the surface of cells was negatively charged, the two mobilities had opposite directions in a field. Fluorescent GFP-expressing E. coli cells moved rapidly in the PDMS channel from the cathode to the anode as a result of the electrophoretic mobility of cells overcoming that of EOF.

The durations of exposure to current were significantly longer than the pulse durations commonly used in electroporation by eletropulsation (˜1-20 ms). Cell viability was not adversely affected by a low field (<300 V/cm) with a long duration (for example, 300 V/cm for 6 seconds or 88 V/cm for 32 seconds in the wide sections of a configuration B device). On the other hand, when the field strength was 2400 V/cm (higher than the threshold of 1500 V/cm), the cell membrane was completely disintegrated within about 400 MS.

Lowering of the threshold was probably related to the longer duration for cells to be exposed to the lysis field in the designed device. This is consistent with the concept that higher field strength would be required to lyse the cells when the duration of the DC field is shorter (Han et al., 2003, Anal. Chemistry 75:3688-3696). The field in the wide sections E₁ had little effect on cell viability. The electroporation and the loss of cell viability occurred in the narrow section.

The amount of time needed for the cells to flow through the narrow section in different configurations was calculated, based on the velocity values and the lengths of the narrow section in different configurations. In FIG. 9, the field strength values were calculated using Equations (1) and (2). FIG. 9 shows: (a) the velocity of cells in the narrow section under various field strengths E2; (b) the duration of stay in the narrow section; (c) the velocity of cells in the wide sections under various field strengths E₁; (d) the duration of stay in the wide sections.

FIG. 9(a) shows that the velocity of cells increased with higher field strength in the narrow section in devices of all three configurations. The difference in the velocity among the three configurations was possibly related to the drag force exerted by the walls on the fluid and cells. Such effects could be dependent on the dimensions of the narrow section.

As can be seen in FIG. 9(b), the duration ranged from 300-500 ms when the lower section field strength E₂ was around 500 V/cm. Shown in FIGS. 9(c) and (d) is the velocity and the duration of stay of cells in the wide sections in devices with different configurations.

In general, the field strength in the wide sections (E₁) was significantly lower than the one in the narrow sections (E₂). In the experiments, only E₁ in configuration C went up to 1000 V/cm due to the low W₁/W₂ (˜2). In configurations A and B, E₁ were in the range of 70-300 V/cm. There were two wide sections (the entry and the exit) in the design. When E₂ was higher than 2000 V/cm, there were no fluorescent cells in the exit wide section due to the complete loss of intracellular materials. In these cases, the velocity of cells was determined based on images of cells in the entry section.

Measuring the velocity of cells in more than one wide section, there was no significant difference between the velocity in the wide section at the entry side of the channel and the velocity at the exit side, even when the field strength E₂ was higher than the threshold and cell lysis occurred during the process (data not shown). As shown in FIG. 9(c), the velocity of cells in the wide sections increased with higher field intensity. The duration of exposure was in the range of 6-45 seconds for the devices with configurations A and B. The duration was significantly shorter in devices with configuration C due to the higher magnitude for E₁, ranging from 1 to 20 seconds.

Other factors might have minor contributions to the loss of cell viability during the process. First, although a buffer with low ionic strength was used and the current was generally very low (<12 μA for configurations A and B and <50 μA for configuration C), Joule heating could still play a role in the process. Joule heating can be particularly detrimental if subsequent assays after cell lysis will be carried out based on proteins which are sensitive to high temperature. Joule heating can be suppressed by using buffers with non-ionic ingredients which still keep the desired osmolarity. Second, a minor degree of electrolysis of water might affect pH in the buffer. This can be prevented by constantly flowing fresh buffer in and out of the reservoirs.

Joule heating and pH change might affect the performance of the device. Accordingly, the methods described here might vary when applied to different applications. For example, the cell velocity may be controlled by the applied electric field. A pressure-driven controlled flow may be added to enable more precise and separate control of the velocity of cells and the field strength.

### Electroporation of Mammalian Cells

For mammalian cells applications, a microfluidic device as shown in FIG. 1, with dimensions of the narrow section slightly larger than a single mammalian cell, was fabricated. The depth and the width of the narrow section were around 30 and 40 μm, respectively. The length of the narrow section was 500 μm. The E₂/E₁ (W₂/W₁) ratio was about 7. The reason for choosing these dimensions was the size of the cells that were electroporated. The fluidic device and method were tested with both Chinese hamster ovary (CHO-K1) and Human colon adenocarcinoma grade II cell line (HT-29) cells.

Pressure driven flow generated by a syringe pump (Harvard Apparatus) was used to control the velocity of cells. The cells were flowing through the microfluidic channel under a pressure and cells passed the narrow section one by one. In the meantime, an electric field was present between the two reservoirs. A hypotonic buffer was used, consisting of 10 mM phosphate, 3 mM HEPES, 125 mM sucrose and 0.05% Tween 20.

The size change on a number of cells was followed. The size and the morphology of cells changed at the entrance of the narrow section when E₂ was high enough, above the threshold for electroporation (electropermeabilization). The diameter of CHO-K1 cells expanded by about 9% when the field in the narrow section E₂ was 150 V/cm, about 27% when E₂ was 200 V/cm, and about 41% when E₂ was 300 V/cm. Similar results were obtained with HT-29 cells, where the expansion was about 46% when E₂ was 300 V/cm.

### Mammalian Cell Lysis Under Constant DC Voltage

The influence of electroporation on cell lysis was tested in some experiments. Chinese Hamster Ovary (CHO-K1) cells were cultured in DMEM medium containing 10% fetal bovine serum (FBS), 100 units of penicillin and 100 μg/ml of streptomycin. They were split every 2-3 days with a ratio from 5:1 to 8:1 to maintain them in the log phase. When confluence was reached, cells were detached from the culture flask using Trypsin-EDTA and then centrifuged at 300×g for 10 min to remove the medium and Trypsin.

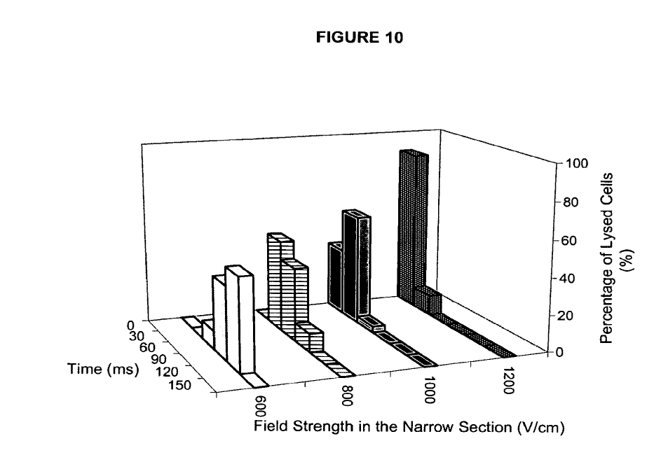

Cell lysis was monitored when the field intensity was in the range of 600-1200 V/cm. FIG. 10 is a graph depicting the percentage of cells lysed during the intervals between imaged frames. Different electric field E₂ intensities in the narrow section of the channel were used. Each curve was obtained based on a sample size of at least 30 cells.

When E₂ was 600 V/cm or higher, 100% of the cells were lysed within 150 ms after entering the narrow section of the flow channel. When E₂ was between 400 and 600 V/cm, cell lysis often did not happen or happened after a longer duration for a given cell. The percentage of cells lysed within each elapsed frame (the interval between frames was 30 ms) was enumerated at different E₂ values (600, 800, 1000, and 1200 V/cm). The onset of release of intracellular materials was considered an indicator of cell lysis.

FIG. 10 shows that the average time for lysis to occur shifted to the shorter end when E₂ increased. More than 90% of the cells were lysed within 30 ms when E₂ was 1200 V/cm. Based on the data shown in FIG. 10, by controlling the strength of the electric field strength in the narrow section and the amount of time that the cells spend in the narrow section, it is possible to control the relative amount of lysed cells. Accordingly, by designing appropriate cross-sectional areas for the narrow section and controlling the electric field strength in the narrow section, it is possible to control the relative amount of lysed cells.

### Electroporation and Viability of Eukaryotic Cells

The influence of electroporation on cell viability was tested in some experiments. Chinese Hamster Ovary (CHO-K1) cells were cultured in DMEM medium as described above. When confluence was reached, cells were detached from the culture flask using Trypsin-EDTA and then centrifuged at 300×g for 10 min to remove the medium and Trypsin.

The fluidic device for delivering SYTOX Green into CHO-K1 cells consisted of two wide channels and one narrow channel alternated (sandwiched in between) the two wide channels (see FIGS. 3 and 5). The width of the narrow section and the width of the wide sections were 62.5 μm and 500 μm, respectively, and the lengths of the narrow and wide sections were 1.5 mm and 1 mm, respectively.

Membrane-impermeant exogenous molecules were introduced into cells during electroporation. SYTOX green nucleic acid stain (MW ˜600, 504/523 nm, Molecular Probes, Eugene, Oreg.) is a green-fluorescent nuclear and chromosome counterstain that is impermeant to live cells and yields >500 fold fluorescence intensity enhancement upon nucleic acid binding. In this experiment, cells were harvested and then centrifuged to remove the medium. They were re-suspended in electroporation buffer (10 mM phosphate buffer, 250 mM sucrose, pH 7.4) with a concentration of 2×10⁶ cells/ml.

Two separate sets of tests were done. In the first set, SYTOX green was added to the cell sample in the electroporation buffer to create a concentration of 1 μM before the sample was delivered into the device for electroporation. The cells were immediately transferred to a 96-well plate and then centrifuged at 300×g for 10 minutes to make them settle to the bottom for observation. The fluorescent cells and the total cell population were enumerated.

In the second set, the cell sample was delivered into the device and electroporated first. Cells collected from the receiving reservoir were added to 100 μl of fresh medium in the 96-well plate immediately after the electroporation. SYTOX green was added to the cell sample 1 hour after the electroporation to achieve the same final concentration (1 μM). The fluorescent and non-fluorescent samples within a population of at least 1,000 cells were enumerated 1.5 hours after the electroporation under a microscope.

The percentage of permeabilized cells together with dead cells among the entire population was obtained from the first set of experiments. The second set of experiments revealed the cell death rate during electroporation. The difference between the two sets of experiments reflects the percentage of cells that were electropermeabilized with preserved viability.

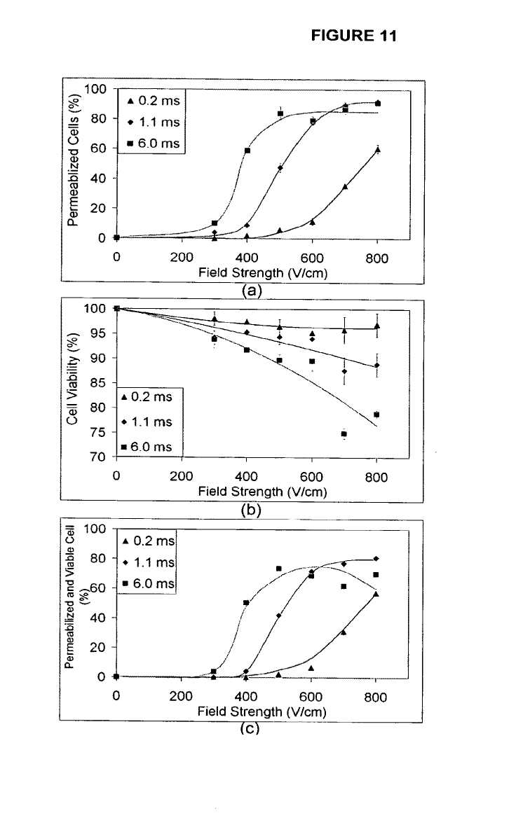

FIG. 11 depicts graphs showing the effects of electric field strength in the narrow section of the channel on CHO-K1 cell permeability and viability, as established via delivery of SYTOX Green into the cells. The legends indicate the time (ms) of exposure of cells to the high field strength inside the narrow section.

### Electrotransfection

Chinese Hamster Ovary (CHO-K1) cells were cultured in DMEM medium containing 10% fetal bovine serum (FBS), 100 units of penicillin and 100 μg/ml of streptomycin. The harvested cell pellet was resuspended in electroporation buffer (10 mM phosphate buffer and 250 mM sucrose) containing 40 μg/ml of pEFGP-C1 plasmid and incubated on ice for at least 5 min before electroporation.

To control the time of exposure of cells to the high field strength in the narrow section, cells were dispensed in the fluidic device by a syringe pump. The amount of time that the cells were exposed to the high field strength was determined by the cell velocity and the length of the channel. A set of separate experiments was conducted to determine the cell velocity and the results showed that the effect of the electric field on the cell velocity was trivial. The duration of field strength was thus directly converted from the infuse rate of the syringe pump. Immediately after electroporation, samples were collected from the receiving reservoir and then transferred to the 96-well plate which was filled with fresh DMEM medium for incubation at 37° C. for 24 hours and 48 hours to observe the cells' viability and transfection rate, respectively. The transfection rate represented the percentage of transfected cells among the viable cells.

To investigate the effects of channel configurations on transfection, two different designs of fluidic devices were used: one design resulted in single pulse-like field strength (single narrow section sandwiched between two wide sections; see FIG. 3). The wide sections and narrow sections were 62.5 μm and 500 μm wide, respectively. The lengths of each wide and narrow section were 1 mm and 1.5 mm. In this configuration, the electric field strength in each narrow section was about 300-800 V/cm. The other configuration enabled exposure of the cells to multiple pulses-like environments (six wide sections with alternated five narrow sections; see FIG. 4). The wide sections and narrow sections were 62.5 μm and 500 μm wide, respectively. The lengths of each wide and narrow section were 200 μm and 500 μm. In this configuration, the electric field strength in each narrow section was about 300-800 V/cm.

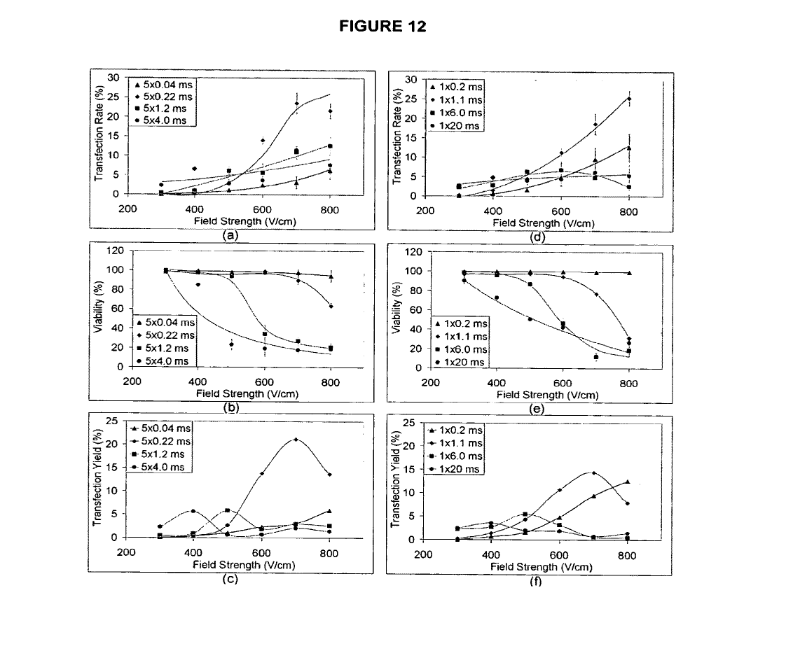

Transfection of CHO-K1 cells was achieved under a variety of conditions. The effects of pulse configurations, strength and duration of electric field on the transfection of CHO-K1 cells are shown in FIG. 12. Panels (a), (b), and (c) show data obtained from channels with multiple pulse-like design (multiple narrow sections), while panels (d), (e), and (f) were obtained from channels with single pulse-like field strength (single narrow section). The sample size ranged from 1000 to 3000 cells for each data point. The legends indicate the number and duration of the high field strength that cells exposed to when flowing through the device. For example, 5×0.04 ms means that cells experienced 5 narrow sections and the duration in each of them was 0.04 ms.

### Cell Fusion Under Constant Dc Voltage

The cells were first conjugated using biotin-streptavidin. Electrofusion was then performed by passing the cells through a microfluidic channel with geometric variation under constant DC voltage. Processing was carried out at single cell pair level.

General information about PDMS microfluidic chip fabrication, culture of CHO-K1 cells, and the application of phase contrast and fluorescence microscopy was provided in the inventors' publications (Wang and Lu, 2006, Anal. Chem. 78: 5158-5164; Wang and Lu, 2006, Biotechnology and Bioengineering, 001:10.1002/bit.21066, in press; Wang et al., 2006, Biosensors and Bioelectronics, D01:10.1016/j.bios.2006.01.032, in press). The excitation and emission from cells labeled with calcein AM or SYTOX (Molecular Probes, Eugene, Oreg.) were filtered by a fluorescence filter cube (exciter HQ480/40, emitter HQ535/50, and beam splitter Q5051 p, Chroma technology, Rockingham, Vt.). The excitation and emission from Hoechst 33342 (Molecular Probes, Eugene, Oreg.) labeling were filtered by a different filter cube (exciter D350/50, emitter D460/50, and beam splitter 400dclp, Chroma technology, Rockingham, Vt.).

As shown in FIGS. 3-5, an electrofusion device consisted of a microfluidic channel with narrow and wide sections. Devices with one or five narrow sections were tested in this work.

Modeling of the electric field intensity in a microfluidic structure with alternated wide and narrow sections when a DC voltage is established across the channel was performed. The modeling suggests that the field strength at the center of the narrow section is around 9.7 times higher than the field strength in the bulk of the wide sections (at least 200 μm away from the narrow section). This number is roughly the ratio between the width in the wide section(s) and the one in the narrow section.

Modeling of the electric field in the device was done applying the Conductive Media DC model from Comsol 3.2 (COMSOL, Inc., Burlington, Mass.) to model the electric field distribution. Assuming there is no ion concentration gradient in the flowing fluid carrying the current, Ohm's law was used for current density calibration,

V(−σ∇V)=0 (4)

where σ is the conductivity (Sm⁻¹), V is the voltage. For the buffer system 1 S/m was used as the value of σ. “Electric potential” option was selected as the boundary condition for the inlet and outlet in the software. The walls were considered as electrically insulated.

In one experiment, a single narrow section was alternated with (sandwiched between) two wide sections. The narrow section was 50 μm long and 40 μm wide. Each of the two wide sections had a width of 400 μm. The depth of the channels was uniformly 33 μm. The total length of the channel was 8.2 mm. In a different experiment, a fluidic device with five narrow sections alternated with six wide sections was used. All dimensions were as above, except that that total length of this device was 13.2 mm.

Cells were harvested by scraping. Cells were not detached using trypsin because cells treated with trypsin would have low affinity to Sulfo-NHS-LC-biotin. The procedure of conjugating cells was similar to what was described in the literature. The cells were first washed by ice-cold PBS buffer (10 mM phosphate buffer, 137 mM NaCl, pH 8.0) twice to remove amine-containing culture medium and cell debris in the solution and then suspended in the same PBS (pH 8.0) buffer at a concentration of 5×10⁷ cells/ml. The cells were then biotinylated by adding Sulfo-NHS-LC-biotin (Pierce, Rockford, Ill.) to a final concentration of 50 μg/10⁶ cells. The cells were incubated at room temperature for 30 min with occasional gentle shaking to prevent cells from aggregation.

After biotinylation, cells were resuspended in PBS buffer (pH=7.4) with 100 mM glycine added for quenching unreacted Sulfo-NHS-LC-biotin residues. One half of the cell sample was transferred to 4° C. water bath for future cell conjugation. The other half of the cell sample was washed by PBS buffer (pH 7.4) twice and then treated for streptavidin coating. Streptavidin in 5 mg/ml stock solution was added to the sample to a concentration of 1 mg/10⁷ cells. The cells were incubated at room temperature for 25 min with gentle shaking. The two cell samples (one coated with biotin and the other coated with biotin-streptavidin) were washed twice and resuspended in electrofusion buffer (1 mM MgSO₄, 8 mM Na₂HPO₄, 2 mM KH₂PO₄, and 250 mM sucrose, pH=7.2) at 5×10⁷ cells/ml before being mixed for cross-linking. The mixed cells were gently concentrated at 300×g for 2-5 seconds until a fraction of the cells precipitated at the bottom of the tube. The sample was then incubated for 15 min. The cell sample was diluted by the electrofusion buffer to 10⁷ cells/ml before the electrofusion experiment.

Typically 50-55% of the cell population was conjugated after these steps, with more than half of them being one-to-one conjugation. To facilitate the observation of cell fusion, in some experiments half of the cells (either biotin coated or biotin/streptavidin coated) were labeled by a fluorogenic dye, calcein AM (Molecular Probes, Eugene, Oreg.). The labeling was done by incubating the cells with calcein AM at a concentration of 1 μg/ml for 10 min.

The microfluidic channel was flushed with electrofusion buffer (1 mM MgSO₄, 10 mM phosphate buffer, and 250 mM sucrose, pH 7.2) for 15 min to condition the channel and remove impurities. The inlet of the channel was connected to a syringe pump (PHD infusion pump, Harvard Apparatus, Holliston, Mass.) through plastic tubing. The pump rate was in the range of 45-225 μl/hr. Considering only the contribution to the cell velocity from the flow rate of the buffer, the durations for cells to be in the narrow section would be 5.3, 2.6, and 1.0 ms when the flow rates are 45, 90, and 225 μl/hr, respectively. However, the actual durations (pulse widths) were shorter than the above numbers and varying with the field intensity, due to the contribution to the cell velocity from the electric field.

A high voltage power supply (PS350, Stanford Research Systems, Sunnyvale, Calif.) was used to generate a direct current (DC) electric field inside the channel. The duration of the electrofusion experiment was 1-3 min until the receiving reservoir contained enough cells for further analysis. Longer processing time may cause significant change in the buffer pH.

Cells were stained by incubation with Hoechst 33342 (1 μg/ml) for 5 min before electrofusion. Cells were transferred to a 96-well plate immediately after electrofusion and observed within 1 hr after the electrofusion under an inverted fluorescence microscope (objective 40×). The number of nuclei per cell and the number of cells containing n nuclei (n as in Equation (5) were counted. Usually about 500 to 1000 cells were enumerated for the calculation of fusion index in one trial and two trials were conducted for one data point.

Two approaches were used to observe the cell fusion. First, half of the cells were labeled with a fluorogenic dye, calcein AM. The other half of the cells was left unlabeled before the chemical conjugation. Cell fusion between labeled cells and unlabeled cells was observed immediately after they flowed through the narrow section. Calcein (the fluorescent derivative of calcein AM) was observed to diffuse into the other half of the fused cell within minutes.

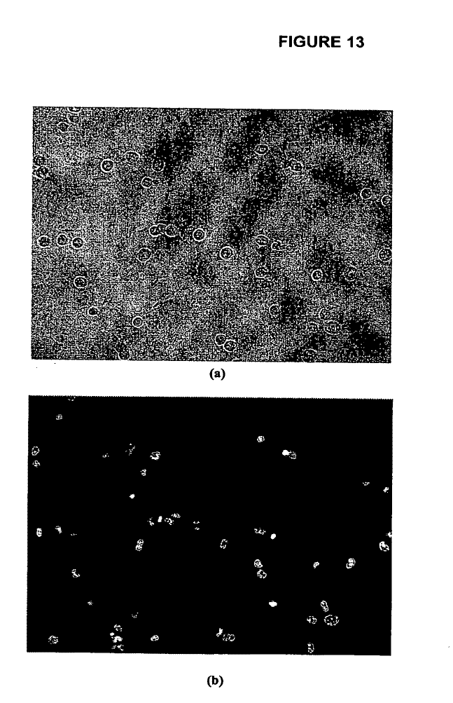

In the second approach, cell nuclei were stained using a nuclear counterstain, Hoechst 33342. The number of nuclei in cells after electrofusion was observed. FIG. 13 shows images of cells processed in a fluidic device for electrofusion. In this experiment the device consisted of one narrow section sandwiched between two wide sections. The electrofusion field was 900 V/cm, and the flow rate was 45 μl/h. Shown in FIG. 13(a) is a phase contrast image of a group of cells processed in the fluidic device. Shown in FIG. 13 (b) is a fluorescent image of the same group of cells as in (a), stained by Hoechst 33342. As can be seen in FIG. 13, a number of cells were observed as containing two or more nuclei. Using the devices and methods of this invention, it was possible to achieve fusion efficiency comparable to that of conventional specialized equipment based on AC alignment and electrical pulses.

The efficiency of cell fusion is characterized using fusion index (FI) which is defined as the fraction of nuclei in polynucleated cells in the total number of nuclei and is calculated using equation (5) below:

$$FI ( \% ) = \frac{{\sum }_{n = 2}^{∞} {nC}_{n}}{{\sum }_{n = 1}^{∞} {nC}_{n}} \times 100 \qquad ( 5 )$$

where Cn is the number of cells containing n nuclei. Two or three nuclei were observed in the vast majority of the fused cells. It needs to be noted that a fraction of the polynucleated cells might occur due to cell division.

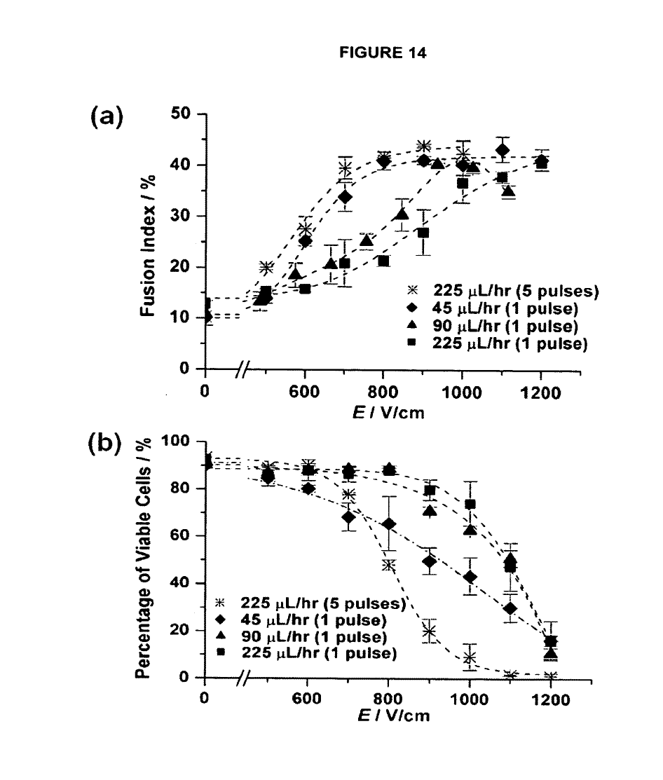

FIG. 14(a) shows the fusion index (among viable cells) at different electrofusion field strengths and flow rates in the single-pulsed and five-pulsed devices. FIG. 14(b) shows the percentage of viable cells measured under the same conditions as in (a). Trend lines are added to guide the eye.

The field in the wide sections, which was substantially lower than the threshold for electric breakdown of the membrane, did not affect the cell viability significantly. The electrofusion field in the narrow section(s) was varied. The duration of exposure or the “pulse width” in the narrow section(s) was also varied by changing the flow rate controlled by the syringe pump. As can be seen in FIG. 14(a), the fusion index was around 10-15% when there was no electric field due to the cell divisions in the cell population.

Depicted in FIG. 14(b) is data showing cell viability after electrofusion as determined using SYTOX exclusion by living cells. Cells were collected from the receiving reservoir (the outlet) immediately after electrofusion and transferred to a 96 well plate with PBS buffer (pH=7.4). The cells were incubated in the PBS buffer with 1 μM SYTOX added for 10 min before the viability was determined (1 hr after electrofusion). Usually about 500 to 1000 cells were enumerated for the calculation of percentile viability in one trial and two trials were conducted for one data point. The viability of cells in general decreased with increasing field strength and pulse width. The use of five-pulsed device created a marked decrease in the cell viability.

In a single-pulsed device (i.e. device with one narrow section), the fusion index increased remarkably when the field strength in the narrow section became higher during the processing. When the field intensity was increased to 1200 V/cm, the fusion index was up to 44% (around 30% after deducting the fraction due to cell division) at all three flow rates. The pulse width made a significant difference when the field intensity was between 600 and 1000 V/cm. The longer pulse width (at lower flow rate) resulted in higher fusion efficiency.

Cell fusion was also carried out in the five-pulsed device (i.e., five narrow sections). The application of multiple pulses improved the efficiency of cell fusion. The five-pulsed device yielded fusion indexes that were consistently higher that those resulting from a single pulse of the same pulse width. The efficiency of cell fusion was comparable to results obtained using conventional pulse generator on the same cell type and similar buffer system.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art, are within the scope of this invention. All publications, patents, and patent applications cited herein are incorporated by reference in their entirety for all purposes.

1. A method of cell electroporation, comprising:
(a) introducing at least one cell into a flow channel of a fluidic device;
(b) subjecting the at least one cell to a constant electric field; and
(c) modifying the intensity of the constant electric field,
where the flow channel is configured such that that upon application of the constant electric field through the flow channel, the electric field intensity in one section of the flow channel is greater than the electric field intensity in another section of the flow channel.

2. The method of claim 1, where modifying the intensity comprises decreasing or increasing the cross-sectional area of the flow channel in the direction of fluid flow.

3. The method of claim 1, where the electric field is generated by constant direct current voltage.

4. The method of claim 1, where the modifying the intensity is such that permeability of the membrane of the at least one cell is increased.

5. The method of claim 2, further comprising the step of delivering a molecule into the cell.

6. The method of claim 2, further comprising the step of lysing the at least one cell.

7. The method of claim 2, further comprising the step of fusing at least two cells.

8. A method of cell electrofusion, comprising:
(a) introducing at least two cells into a flow channel of a fluidic device;
(b) subjecting the at least two cells to a constant electric field; and
(c) modifying the intensity of the constant electric field, such that the strength of the electric field is greater than the electric field intensity threshold for electrofusion of the at least two cells.

9. The method of claim 8, where modifying the intensity comprises decreasing or increasing the cross-sectional area of the flow channel in the fluid flow direction.

10. The method of claim 8, where the constant electric field is generated by constant direct current voltage.

11. The method of claim 1, wherein the constant electric field is applied for at least one minute.

12. The method of claim 8, wherein the constant electric field is applied for at least one minute.
